(19) United States
(12) Patent Application Publication
(10) Pub. No.: US 2025/0099052 A1
(43) Pub. Date: Mar. 27, 2025

(54) PLACEMENT MECHANISM AND FLAT PANEL BOX

(71) Applicant: Shanghai United Imaging Healthcare Co., Ltd., Shanghai (CN)

(72) Inventors: Junxiao Zhang, Shanghai (CN); Bo Shi, Shanghai (CN); Yang Tang, Shanghai (CN); Liyong Qin, Shanghai (CN)

(21) Appl. No.: 18/890,800

(22) Filed: Sep. 20, 2024

(30) Foreign Application Priority Data
Sep. 21, 2023 (CN) 202322577070.0

(51) Int. Cl. A61B 6/00

(52) U.S. Cl. CPC A61B 6/44

(57) ABSTRACT

A placement mechanism and a flat panel box are provided. The placement mechanism includes a body with a mounting surface configured to place a flat panel and a limiting assembly on the mounting surface. The limiting assembly includes at least two first limiting blocks at a first end of the body, a second limiting block at a second end of the body, and a locking portion. The at least two first limiting blocks include a flange extending over the mounting surface along a height direction of the body. The at least two first limiting blocks and the second limiting block are configured to limit a length direction and a width direction of the flat panel. The locking portion is located at the second end and has a locking member with a locking position and an unlocking position configured to lock and unlock a height direction of the flat panel.

### CROSS-REFERENCE TO RELATED APPLICATION

This application claims priority to Chinese patent application No. 202322577070.0, filed on Sep. 21, 2023, titled “PLACEMENT MECHANISM AND FLAT PANEL BOX”, the content of which is hereby incorporated by reference in its entirety.

### TECHNICAL FIELD

The present disclosure generally relates to the field of medical instruments, and in particular, to a placement mechanism and a flat panel box.

### BACKGROUND

At present, an X-ray imaging technology has been developed into a digital X-ray imaging phase, and a flat panel detector (a flat panel for short) is widely used in a clinical field, a seismic field, and the like as a benchmark device of the digital X-ray imaging. The flat plate is divided into a direct type and an intermediate type. A material of the flat plate is hydrogenated amorphous silicon, but the material is easily damaged. To protect the flat panel, some flat panel placement mechanisms have been designed, but these mechanisms still have some problems, such as unreliability and unsatisfactory ergonomics.

The Chinese Patent (Publication No. CN211484625U) discloses a flat panel placement mechanism. A principle of the mechanism is that three corners of a flat panel are limited by a fixed plate, a first card holder, a second card holder, and a card holder base and a stopper, so that the flat panel is fixed. However, the mechanism cannot effectively prevent the flat panel from jumping out in a seismic environment, and when the flat panel needs to be placed or taken, the flat panel should be parallel to the fixed plate, which is not easy to operate.

The Chinese Patent (Publication No. CN202776334U) discloses a locking structure, a flat panel combination apparatus, and an X-ray imaging device. The apparatus can prevent a flat panel from jumping out in the seismic environment. A principle of the apparatus is that the flat panel is fixed by a first fastener, a second fastener, and a locking structure. However, the apparatus needs to be manually operated by a doctor, so that an appearance of the flat panel is easily scratched. At the same time, it requires both hands of the doctor to unlock the locking structure that releases the flat panel, which does not meet ergonomics.

### SUMMARY

According to various embodiments of the present disclosure, a placement mechanism and a flat panel box are provided.

In a first aspect, a placement mechanism having a body is provided. The body has a mounting surface, the mounting surface is configured to place a flat panel, the placement mechanism further includes a limiting assembly disposed on the mounting surface, and the limiting assembly includes a first limiting block, a second limiting block, and a locking portion.

The quantity of the first limiting block is at least two, the body has a first end and a second end relative to each other, and at least two first limiting blocks are distributed at the first end.

The second limiting block is located at the second end, and the first limiting block and the second limiting block are configured to perform limiting along at least one of a length direction, a width direction, or a height direction of the flat panel.

The locking portion is located at the second end, the locking portion has a locking member, the locking member is configured to lock and unlock the flat panel, respectively.

In some embodiments, the locking portion further includes an adjusting member configured to adjust a friction force between the locking member and the body.

In some embodiments, the body includes a locking base, the lock member is rotatably connected to the locking base, the locking portion further includes a first spring, the adjusting member is connected to the locking member, and the first spring is disposed between the adjusting member and the locking base.

In some embodiments, the limiting assembly further includes an elastic member, the elastic member is disposed on either or both of the first limiting block and the second limiting block, and the elastic member is configured to exert a force on the flat panel along the length direction or the width direction.

In some embodiments, the quantity of the elastic member is at least two, at least two elastic members are distributed on the first limiting block and the second limiting block, respectively, and the at least two elastic members are configured to exert elastic forces on the flat panel along the length direction or the width direction.

In some embodiments, the first limiting block includes a limiting surface, the limiting surface is configured to align limiting with the length direction or the width direction of the flat panel, the limiting surface has a guiding region, and an angle is defined between the guiding region and a mounting direction of the flat panel.

In some embodiments, the placement mechanism further includes a drag reducing portion disposed on the mounting surface and the drag reducing portion has a smooth surface.

In some embodiments, the drag reducing portion is in a strip shape, the quantity of the drag reducing portion is at least two, and at least two drag reducing portions extend along a mounting direction of the flat panel.

In some embodiments, one of the at least two first limiting blocks includes a flange, and the flange extends over the mounting surface along a height direction of the body.

In some embodiments, the locking member has a locking position and an unlocking position, and the locking position and the unlocking position are configured to lock and unlock the flat panel along the height direction, respectively.

In a second aspect, a flat panel box is further provided. The flat panel box includes the above placement mechanism, and the flat panel box further includes a bottom plate and a charging assembly.

The placement mechanism is mounted on the bottom plate.

The charging assembly includes a charging head, a second spring, and a mounting base, the charging head is configured to charge the flat panel, the mounting base is fixed on the bottom plate, and the charging head is floating connected to the mounting base by the second spring.

In some embodiments, the bottom plate includes a guiding rail, the placement mechanism includes a guiding portion that cooperates with the guiding rail, and a direction of insertion of the charging head is the same as a direction of extension of the guiding rail.

### BRIEF DESCRIPTION OF THE DRAWINGS

In order to more clearly illustrate the technical solutions in the embodiments of the present disclosure or the related technology, the accompanying drawings to be used in the description of the embodiments or the related technology will be briefly introduced below, and it will be obvious that the accompanying drawings in the following description are only some of the embodiments of the present disclosure, and that, for one skilled in the art, other accompanying drawings can be obtained based on these accompanying drawings without putting in creative labor.

FIG. 1 is a schematic diagram of a flat panel box in an embodiment of the present disclosure.

FIG. 2 is a schematic diagram of a placement mechanism in an embodiment of the present disclosure.

FIG. 3 is a schematic diagram of a first limiting block in an embodiment of the present disclosure.

FIG. 4 is an enlarged diagram of a first limiting block of FIG. 2.

FIG. 5 is an enlarged diagram of a second limiting block of FIG. 2.

FIG. 6 is a schematic diagram of a placement mechanism in a locked state in an embodiment of the present disclosure.

FIG. 7 is an enlarged diagram of a locking portion of FIG. 6.

FIG. 8 is a schematic diagram of a placement mechanism in an unlocked state in an embodiment of the present disclosure.

FIG. 9 is an enlarged diagram of a locking portion of FIG. 8.

FIG. 10 is a schematic diagram of an internal structure of a locking portion in an embodiment of the present disclosure.

FIG. 11 is a schematic diagram of a bottom plate in an embodiment of the present disclosure.

FIG. 12 is a schematic diagram of a charging assembly in an embodiment of the present disclosure.

In the figures, 100 represents a flat panel box, 1 represents a placement mechanism, 11 represents a body, 111 represents a mounting surface, 112 represents a first end, 113 represents a second end, 114 represents a locking base, 121 represents a first limiting block, 1211 represents a flange, 1212 represents a guiding region, 1213 represents a limiting region, 122 represents a second limiting block, 123 represents a locking portion, 1231 represents a locking member, 12311 represents a handle, 12312 represents a locking shaft, 1232 represents an adjusting member, 1233 represents a first spring, 1234 represents a buckle cover, 1235 represents a friction plate, 124 represents an elastic member, 13 represents a drag reducing portion, 2 represents a bottom plate, 21 represents a guiding rail, 22 represents a positioning block, 3 represents a charging assembly, 31 represents a charging head, 32 represents a second spring, 33 represents a mounting base, 34 represents a guiding base, 35 represents a connecting plate, 200 represents a flat panel, and L represents a mounting direction of the flat panel.

### DETAILED DESCRIPTION OF THE EMBODIMENT

The technical solutions in the embodiments of the present disclosure will be described clearly and completely in the following in conjunction with the accompanying drawings in the embodiments of the present disclosure, and it is obvious that the described embodiments are only a part of the embodiments of the present disclosure, but not all of the embodiments. Based on the embodiments in the present disclosure, all other embodiments obtained by one skilled in the art without making creative labor fall within the scope of protection of the present disclosure.

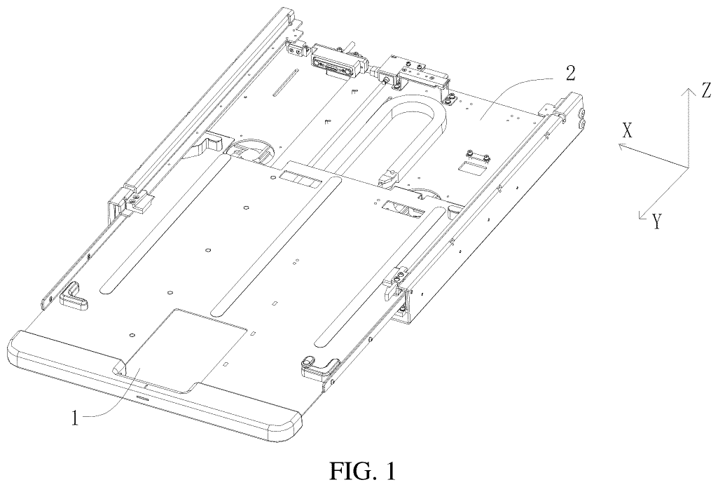

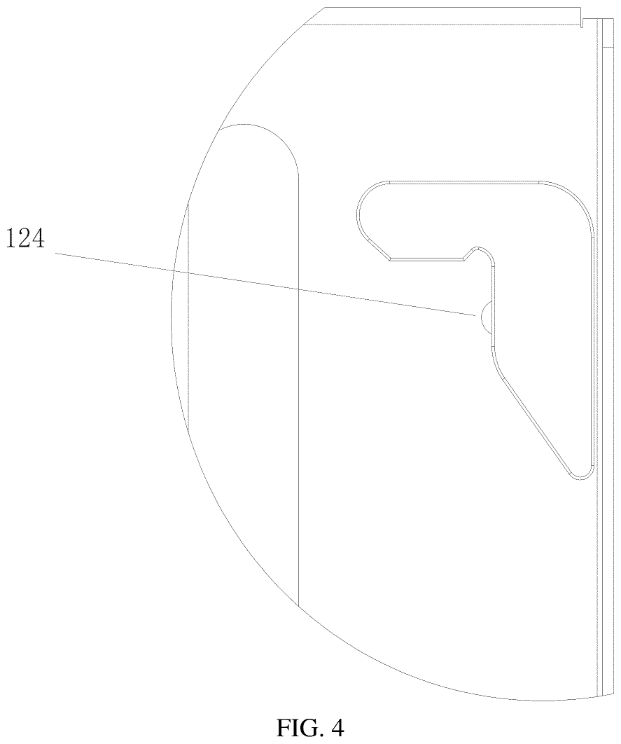

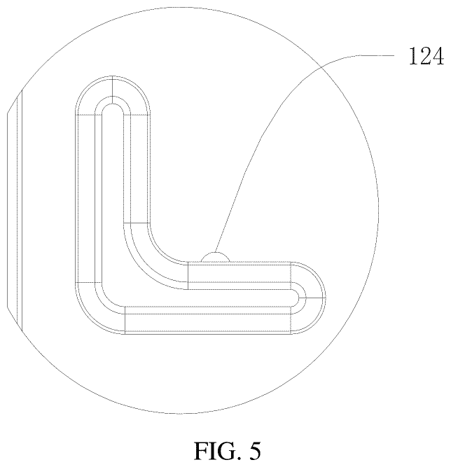

Referring to FIG. 1 to FIG. 12, a flat panel box is provided in the present embodiment. The flat panel box includes a placement mechanism 1. The placement mechanism 1 has a body 11, the body 11 has a mounting surface 111, the mounting surface 111 is configured to place a flat panel 200, the placement mechanism 1 further includes a limiting assembly disposed on the mounting surface 111, and the limiting assembly includes a first limiting block 121, a second limiting block 122, and a locking portion 123. The quantity of the first limiting block 121 is at least two, the body 11 has a first end and a second end relative to each other, and at least two first limiting blocks 121 are distributed at the first end 112. The second limiting block 122 is located at the second end 113, and the first limiting block 121 and the second limiting block 122 are configured to limit at least one of a length direction, a width direction, or a height direction of the flat panel 200. The locking portion 123 is located at the second end 113, and the locking portion 123 is configured to lock and unlock the flat panel 200 along the height direction, respectively.

In the present embodiment, the placement mechanism 1 may perform limiting along at least one of the length direction, the width direction, or the height direction of the flat panel 200 by the limiting assembly including the first limiting block 121 and the second limiting block 122. The locking portion 123 may be designed to allow a locking member 1231 to rotate relative to the body 11, so that the user can easily lock or unlock the flat panel 200 along the height direction without using double hands, thereby improving operation convenience. The design may enhance placement reliability of the flat plate 200, avoid jumping out in a seismic environment or other vibration environment, improve operation convenience, and meet ergonomics.

Specifically, in the present embodiment, the width direction may be an X direction, the length direction may be a Y direction, and the height direction may be a Z direction.

In the present embodiment, the quantity of the first limiting block 121 is at least two, the quantity of the second limiting block 122 may be one, the first end 112 and the second end 113 may be distributed along the length direction, and the at least two first limiting blocks 121 and the second limiting block 122 may be distributed along the length direction on the first end 112 and the second end 113, respectively. In another alternative embodiment, the first end 112 and the second end 113 may also be distributed along the width direction, and the at least two first limit blocks 121 and the second limit block 122 may be distributed along the width direction on the first end 112 and the second end 113, respectively.

In some embodiments, one of the at least two first limiting blocks 121 may include a flange 1211, and the flange 1211 may extend over the mounting surface 111 along a height direction of the body 11. It may effectively restrict movement and lifting of the flat panel 200 along the height direction.

In some embodiments, the locking portion 123 may have a locking member 1231, the locking member 1231 has a locking position and an unlocking position, and the locking position and the unlocking position are configured to lock and unlock the flat panel 200 along the height direction, respectively. The locking member 1231 can rotate relative to the body 11 to switch between the locking position and the unlocking position.

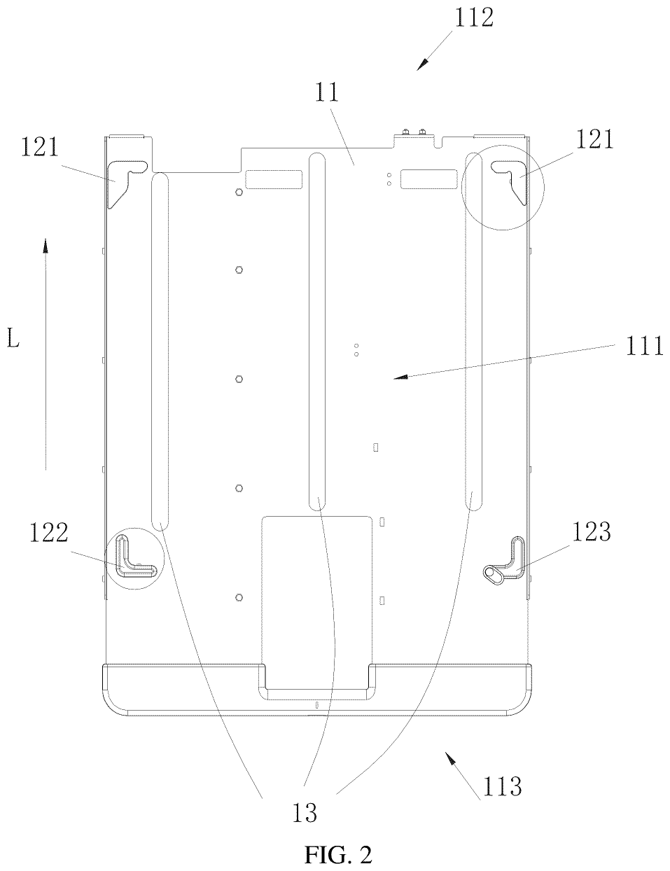

Referring to FIG. 2 to FIG. 5, the limiting assembly may further include an elastic member 124, the elastic member 124 may be disposed on both of the first limiting block 121 and the second limiting block 122, and the elastic member 124 is configured to exert a force on the flat panel 200 along the length direction and the width direction.

The limiting assembly may exert the force on the flat panel 200 along the length direction or the width direction by setting the elastic member 124, which may further enhance stability of the flat panel 200 on the mounting surface 111, so as to ensure that the flat panel 200 is securely fixed at a specified position, so that displacement or shaking may not be easily generated. In addition, the elastic member 124 may be designed so that the limiting assembly may have a certain elasticity, and the limiting assembly may be adaptively adjusted according to different sizes and shapes of the flat panel 200. In this way, the placement mechanism 1 may be applicable to different types of the flat panel 200. In another alternative embodiment, the elastic member 124 may be disposed on the first limiting block 121 or the second limiting block 122.

Specifically, the elastic member 124 may be made of a spring steel, a rubber, or other elastic material to provide an elastic force applied to the flat panel 200.

In the present embodiment, the quantity of the elastic member 124 may be two, two elastic members 124 may be distributed on the first limiting block 121 and the second limiting block 122, respectively, and the two elastic members 124 are configured to exert elastic forces on the flat panel 200 along the length direction and the width direction. In another alternative embodiment, the quantity of the elastic member 124 may also be more than two to enable a limiting force on the flat panel 200 more uniform. Distribution of a plurality of elastic members 124 may enable elastic forces exerted on the flat panel 200 to be uniformly distributed along the length direction and the width direction, so that the flat panel 200 may be adequately limited in both directions, thereby implementing effective constraint on the flat panel 200, facilitating maintaining a stable position of the flat panel 200 in the placement mechanism 1, and preventing the flat panel 200 from being displaced or dislodged in a moving or vibrating environment. In addition, the elastic force may be set to accommodate different sizes of the flat panel 200, and the placement mechanism 1 may be more versatile and flexible.

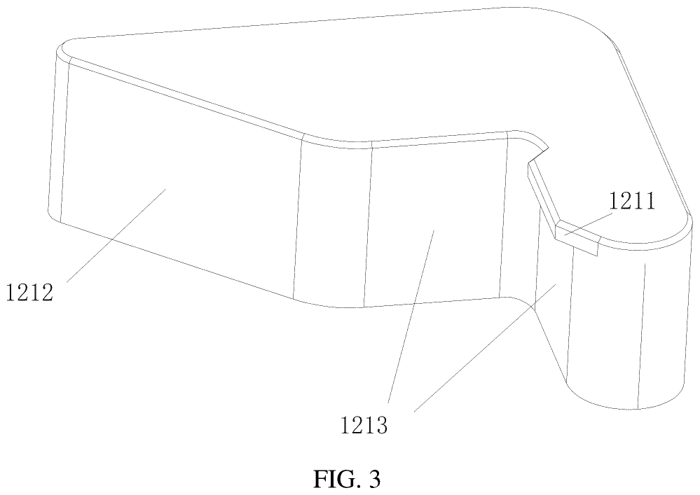

Referring to FIG. 2 and FIG. 3, the first limiting block 121 may include a limiting surface, the limiting surface is configured to align limiting with the length direction or the width direction of the flat panel 200, the limiting surface may have a guiding region 1212, and an angle may be defined between the guiding region 1212 and a mounting direction of the flat panel 200.

The guiding region 1212 may be an inclined surface portion of the limiting surface along the mounting direction of the flat panel 200. When the flat panel 200 is inserted into the placement mechanism 1, the guiding region 1212 may guide the flat panel 200, which may assist the user in accurately locating and placing the flat panel 200 on the limiting surface. The existence of the guiding region 1212 may enable a process of placing the flat panel 200 to be simpler and more accurate, and improve placement efficiency and accuracy.

Referring to FIG. 2, the placement mechanism 1 may further include a drag reducing portion 13 disposed on the mounting surface 111 and the drag reducing portion 13 may have a smooth surface.

The drag reducing portion 13 may be added to the placement mechanism 1 with a smooth surface. The smooth surface may effectively reduce wear and scratch between the flat panel 200 and the mounting surface 111, protect a surface of the flat panel 200 from being scraped or damaged, and effectively reduce friction between the flat panel 200 and the mounting surface 111, so that the flat panel 200 may be more smooth in a placement and removal process, and convenience and stability of the placement mechanism 1 may be improved.

Specifically, the drag reducing portion 13 may be in a strip shape, and the quantity of the drag reducing portion 13 may be at least two, and at least two drag reducing portions 13 may extend along the mounting direction of the flat panel 200.

In the present embodiment, the quantity of the drag reducing portion 13 may be three, and three drag reducing portions 13 may be distributed at equal intervals. In another alternative embodiment, the quantity of the drag reducing portion 13 may be another number equal to or more than two. The drag reducing portions 13 may be in a strip shape and extend along the mounting direction of the flat panel 200, so that a contact region between the flat panel 200 and the mounting surface 111 may be covered more comprehensively. This design may effectively reduce an overall friction force, so that the flat panel 200 may be more smooth and stable in the placement and removal process. In addition, the drag reducing portions 13 may be disposed so that resistance reduction may be distributed evenly, so as to ensure that the flat panel 200 may be supported uniformly and placed stably, thereby avoiding deformation or damage caused by excessive local pressure.

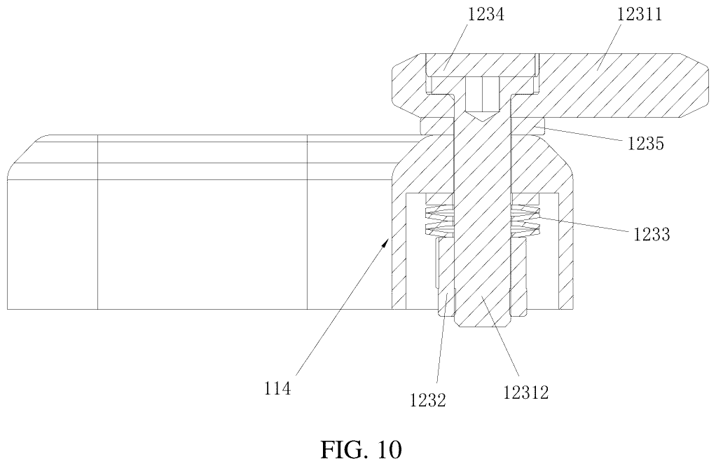

Referring to FIG. 10, the locking portion 123 may further include an adjusting member 1232 configured to adjust a friction force between the locking member 1231 and the body 11. The friction force may be adjusted by the adjusting member 1232, so that the locking member 1231 may obtain sufficient fastening force in the locking position, and it is ensured that the flat panel 200 is securely fixed on the mounting surface 111. This friction force can resist an external force action in the seismic environment or other vibration environment, and prevent the locking member 1231 from unlocking accidentally due to an external force, which may meet an ergonomic requirement and improve applicability and reliability of the placement mechanism 1 for the flat panel 200.

Referring to FIG. 10, the body 11 may include a locking base 114, the locking member 1231 may be rotatably connected to the locking base 114, the locking portion 123 may further include a first spring 1233, the adjusting member 1232 may be threadably connected to a locking shaft 12312, and the first spring 1233 may be disposed between the adjusting member 1232 and the locking base 114. When the adjusting member 1232 rotates, a certain spring force may be generated due to a function of the spring. The spring force may be transmitted to the locking member 1231 by the locking shaft 12312, the locking member 1231 may be compressed, and a force of compression may affect a friction force between the locking member 1231 and the body 11. A degree of loosening of the adjusting member 1232 may change a size of the spring force, so that a friction force of the locking member 1231 may be adjusted. In other words, a certain elastic force is provided, so that a rotation of the locking shaft 12312 has a certain damping force, so as to ensure that a handle 12311 is not too loose or tightened during operation. The user may adjust, according to an actual requirement, the friction force between the locking member 1231 and the body 11, so as to control stability and fastness of the flat panel 200. When a stronger locking force is required, the adjusting member 1232 may rotate to increase pressure of the spring. On the contrary, when a relatively light locking force is required, the pressure of the spring may be reduced. This adjustable feature may enable the operation to be more flexible and convenient, while ensuring safe and stable placement of the flat panel 200.

Specifically, the locking member 1231 may be threadably connected to the locking shaft 12312, the locking member 1231 may rotate to switch between the locking position and the unlocking position, and the locking part 123 further includes a latch 1234, which is located above the locking member 1231, so as to prevent the locking shaft 12312 from being exposed. The friction plate 1235 is located between the body 11 and the locking member 1231. A function of the friction plate 1235 is to increase a friction force between the body 11 and the locking member 1231, so as to increase a fastening force of the locking member 1231 on the plate 200 when the locking position is locked, so as to keep the plate 200 firmly fastened.

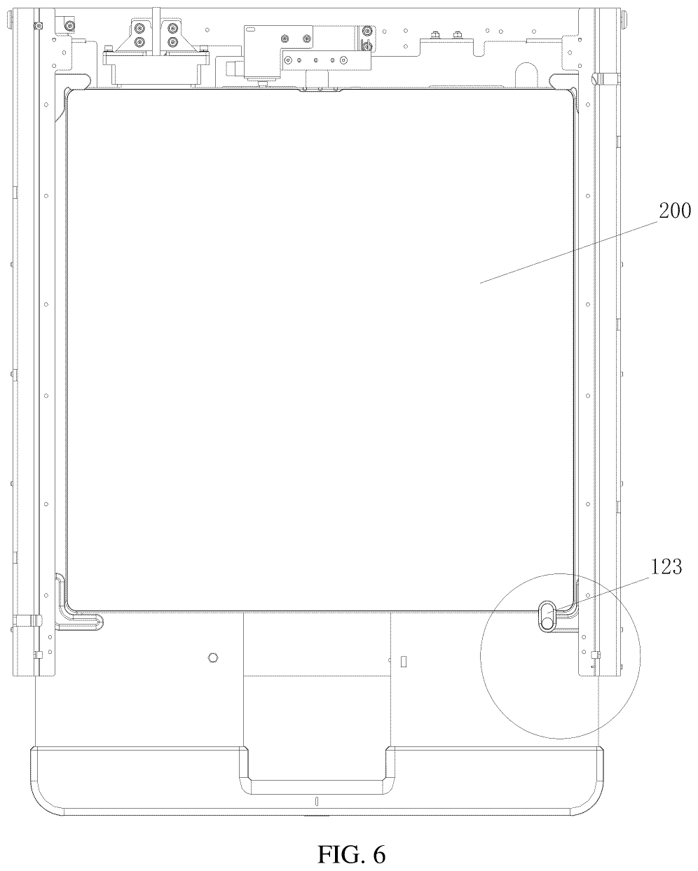

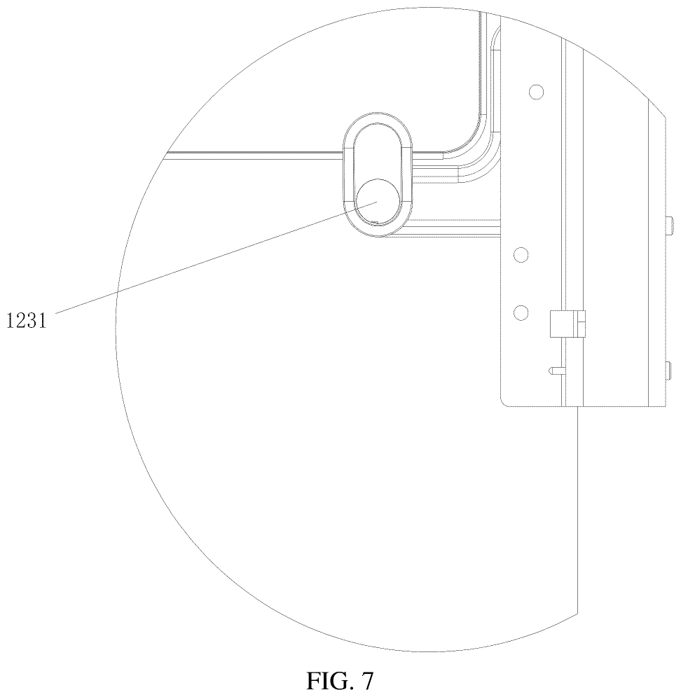

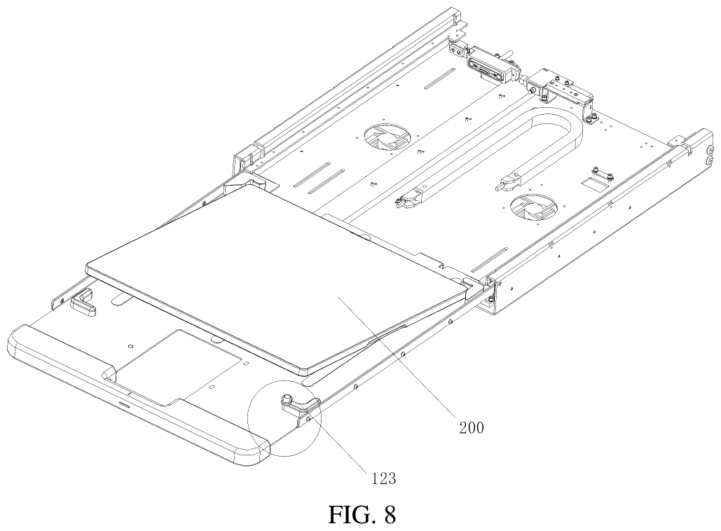

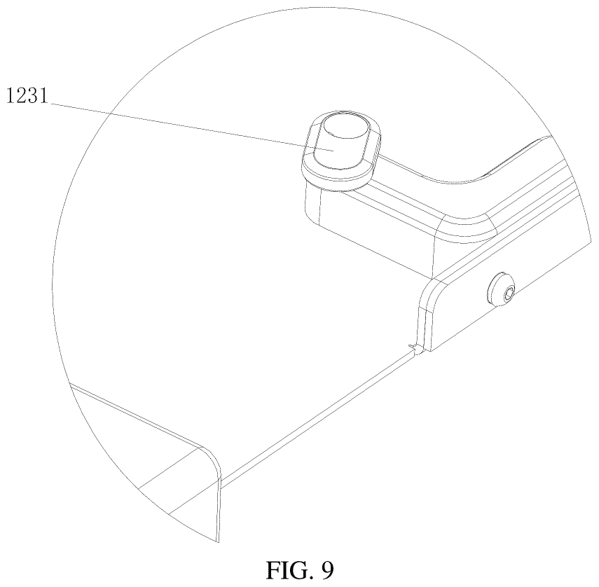

Specifically, the locking member 1231 may include a handle 12311 and a lock shaft 12312. The lock shaft 12312 may be rotatably connected to the lock base 114. The user may adjust a locking state of the locking portion 123 by the handle 12311. Referring to FIG. 6 and FIG. 7, when the handle 12311 is in an internal rotation state, the locking member 1231 may be in a locking position, and the flat panel 200 may be limited along the height direction by the locking member 1231. Referring to FIG. 8 and FIG. 9, when the handle 12311 is in an external rotation state, the locking member 1231 may be in an unlocking position, the flat panel 200 may not be limited along the height direction by the locking member 1231, and the flat panel 200 may be freely placed in the placement mechanism 1 or taken out from the placement mechanism 1.

In another embodiment, the locking portion 123 may also be disposed on the second limiting block 122, and the flat panel 200 may also be limited along the height direction.

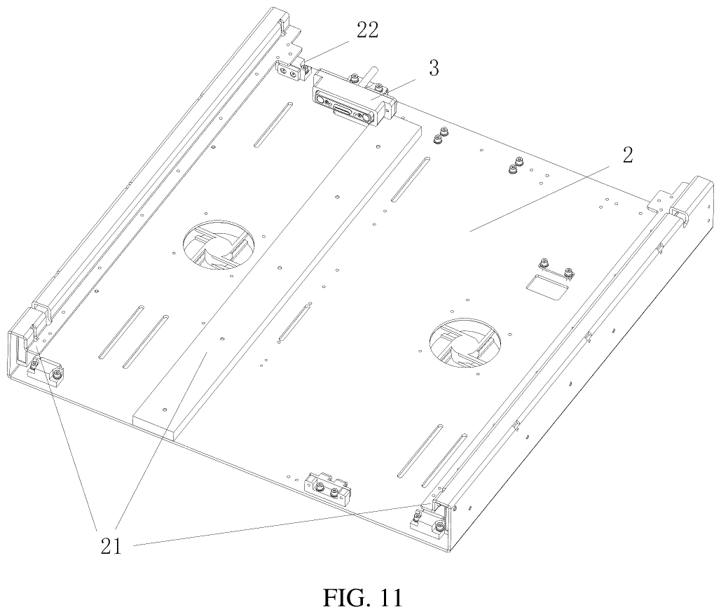

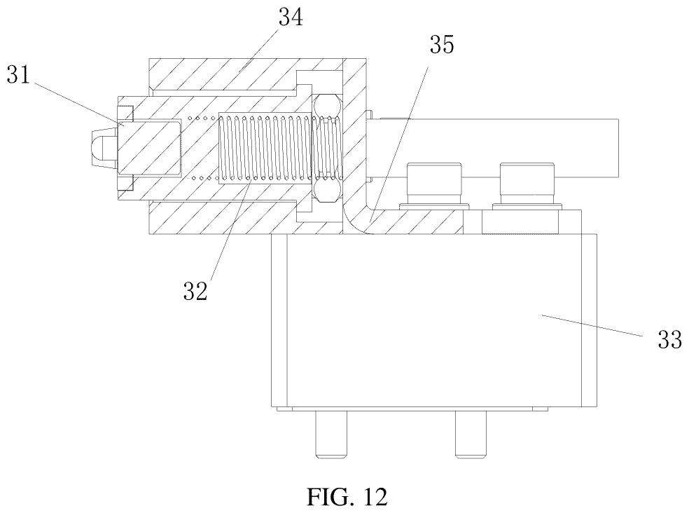

Referring to FIG. 11 and FIG. 12, the flat panel box 100 further includes a bottom plate 2 and a charging assembly 3. The placement mechanism 1 is mounted on the bottom plate 2. The charging component 3 includes a charging head 31, a second spring 32, and a mounting base 33. The charging head 31 is configured to charge the flat panel 200, the mounting base 33 is fixed on the bottom plate 2, and the charging head 31 is floating connected to the mounting base 33 by the second spring 32.

Elasticity of the second spring 32 may allow the charging head 31 to float freely within a certain range, so as to adapt to vibration or direction change of the flat panel 200. When a position of the flat panel 200 changes slightly due to an external force, the charging head 31 may adjust the position automatically according to the elasticity of the second spring 32, and maintain good contact with a plug-in port, so as to maintain a stable electrical connection state. By designing a floating connection of the second spring 32, the flat panel 200 may follow a vibration environment to ensure stability and reliability of the electrical connection state, so that the flat panel box 100 may maintain safe storage and charging of the flat panel 200 in a complex application environment.

Specifically, the charging assembly 3 may further include a guiding base 34 and a connecting plate 35. The guiding base 34 is configured to guide the charging head 31, and the connecting plate 35 is configured to connect the charging head 31 to the mounting base 33. Furthermore, the charging head 31 may include a charging interface.

Specifically, the bottom plate 2 may include a guiding rail 21, the placement mechanism 1 may include a guiding portion (not shown in the figures) that cooperates with the guiding rail 21, and a direction of insertion of the charging head 31 may be the same as a direction of extension of the guiding rail 21. A structure of the guiding portion cooperating with the guiding rail 21 may ensure stable positioning of the placement mechanism 1 inside the flat panel box 100. The direction of insertion of the charging head 31 may be the same as the direction of extension of the guiding rail 21, i.e., the direction of insertion of the charging head 31 may be the same as a sliding direction of the guiding rail 21. This design may simplify a process of insertion of the charging head 31, and enable it more convenient and smoother for the user to place the flat panel 200 and perform charging.

Furthermore, the bottom plate 2 may further include a positioning block 22, the placement mechanism 1 may be connected to the bottom plate 2 by the guiding rail 21, the placement mechanism 1 may move along the guiding rail 21 and be limited by the positioning block 22.

An operating process of the flat panel 200 and the flat panel box 100 may be as follows: The locking member 1231 may be rotated to the unlocking position by the handle 12311, the user may place the flat panel 200 into the flat panel box 100, the flat panel 200 may be in contact with the limiting assembly of the placement mechanism 1, the flat panel 200 may be limited along the length direction, the width direction, and the height direction, the user may rotate the locking member 1231 to the locking position by the handle 12311, with reference to the limiting of the flange 1211, the flat panel 200 may be locked along the height direction, and the flat panel 200 may be firmly fixed at a specified position, so as to avoid jumping out in a seismic environment or other vibration environment. When the flat panel 200 needs to be removed, the user may rotate the locking member 1231 to the unlocking position again by the handle 12311, and the flat panel 200 may be unlocked along a height direction and freely removed from the placement mechanism 1.

The various technical features of the above-described embodiments may be combined arbitrarily, and all possible combinations of the various technical features of the above-described embodiments have not been described for the sake of conciseness of description. However, as long as there is no contradiction in the combinations of these technical features, they should be considered to be within the scope of the present specification.

The above-described embodiments express only several embodiments of the present disclosure, which are described in a more specific and detailed manner, but are not to be construed as a limitation on the scope of the present disclosure. For one skilled in the art, several deformations and improvements can be made without departing from the conception of the present disclosure, all of which fall within the scope of protection of the present disclosure. Therefore, the scope of protection of the present disclosure shall be subject to the attached claims.

What is claimed is:

1. A placement mechanism having a body, wherein the body has a mounting surface, the mounting surface is configured to place a flat panel, the placement mechanism further comprises a limiting assembly disposed on the mounting surface, and the limiting assembly comprises a first limiting block, a second limiting block, and a locking portion;
the quantity of the first limiting block is at least two, the body has a first end and a second end relative to each other, and at least two first limiting blocks are distributed at the first end;
the second limiting block is located at the second end, and the first limiting block and the second limiting block are configured to perform limiting along at least one of a length direction, a width direction, or a height direction of the flat panel; and
the locking portion is located at the second end, the locking portion has a locking member, and the locking member is configured to lock and unlock the flat panel, respectively.

2. The placement mechanism of claim 1, wherein the locking portion further comprises an adjusting member configured to adjust a friction force between the locking member and the body.

3. The placement mechanism of claim 2, wherein the body comprises a locking base, the lock member is rotatably connected to the locking base, the locking portion further comprises a first spring, the adjusting member is connected to the locking member, and the first spring is disposed between the adjusting member and the locking base.

4. The placement mechanism of claim 1, wherein the limiting assembly further comprises an elastic member, the elastic member is disposed on either or both of the first limiting block and the second limiting block, and the elastic member is configured to exert a force on the flat panel along the length direction or the width direction.

5. The placement mechanism of claim 4, wherein the quantity of the elastic member is at least two, at least two elastic members are distributed on the first limiting block and the second limiting block, respectively, and the at least two elastic members are configured to exert elastic forces on the flat panel along the length direction or the width direction.

6. The placement mechanism of claim 1, wherein the first limiting block comprises a limiting surface, the limiting surface is configured to align limiting with the length direction or the width direction of the flat panel, the limiting surface has a guiding region, and an angle is defined between the guiding region and a mounting direction of the flat panel.

7. The placement mechanism of claim 1, wherein the placement mechanism further comprises a drag reducing portion disposed on the mounting surface and the drag reducing portion has a smooth surface.

8. The placement mechanism of claim 7, wherein the drag reducing portion is in a strip shape, the quantity of the drag reducing portion is at least two, and at least two drag reducing portions extend along a mounting direction of the flat panel.

9. The placement mechanism of claim 1, wherein one of the at least two first limiting blocks comprises a flange, and the flange extends over the mounting surface along a height direction of the body.

10. The placement mechanism of claim 1, wherein the locking member has a locking position and an unlocking position, and the locking position and the unlocking position are configured to lock and unlock the flat panel along the height direction, respectively.

11. A flat panel box, comprising the placement mechanism of claim 1, wherein the flat panel box further comprises a bottom plate and a charging assembly;
the placement mechanism is mounted on the bottom plate; and
the charging assembly comprises a charging head, a second spring, and a mounting base, the charging head is configured to charge the flat panel, the mounting base is fixed on the bottom plate, and the charging head is floating connected to the mounting base by the second spring.

12. The flat plate box of claim 11, wherein the bottom plate comprises a guiding rail, the placement mechanism comprises a guiding portion that cooperates with the guiding rail, and a direction of insertion of the charging head is the same as a direction of extension of the guiding rail.

13. The flat plate box of claim 11, wherein the locking portion further comprises an adjusting member configured to adjust a friction force between the locking member and the body.

14. The flat plate box of claim 13, wherein the body comprises a locking base, the lock member is rotatably connected to the locking base, the locking portion further comprises a first spring, the adjusting member is connected to the locking member, and the first spring is disposed between the adjusting member and the locking base.

15. The flat plate box of claim 11, wherein the limiting assembly further comprises an elastic member, the elastic member is disposed on either or both of the first limiting block and the second limiting block, and the elastic member is configured to exert a force on the flat panel along the length direction or the width direction.

16. The flat plate box of claim 15, wherein the quantity of the elastic member is at least two, at least two elastic members are distributed on the first limiting block and the second limiting block, respectively, and at least two elastic members are configured to exert elastic forces on the flat panel along the length direction or the width direction.

17. The flat plate box of claim 11, wherein the first limiting block comprises a limiting surface, the limiting surface is configured to align limiting with the length direction or a width direction of the flat panel, the limiting surface has a guiding region, and an angle is defined between the guiding region and a mounting direction of the flat panel.

18. The flat plate box of claim 11, wherein the placement mechanism further comprises a drag reducing portion disposed on the mounting surface and the drag reducing portion has a smooth surface.

19. The flat plate box of claim 18, wherein the drag reducing portion is in a strip shape, the quantity of the drag reducing portion is at least two, and at least two drag reducing portions extend along a mounting direction of the flat panel.

20. The flat plate box of claim 11, wherein one of the at least two first limiting blocks comprises a flange, and the flange extends over the mounting surface along a height direction of the body.
